(19) United States
(12) Patent Application Publication
(10) Pub. No.: US 2004/0097855 A1
(43) Pub. Date: May 20, 2004

(54) Joint wrap having a specifically disposed aperture

(75) Inventors: Gwen Elaine Page, Morrow, OH (US); Andrew Raymond Cipra, Maineville, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/702,892

(22) Filed: Nov. 6, 2003

Related U.S. Application Data
(60) Provisional application No. 60/424,200, filed on Nov. 6, 2002.

(51) Int. Cl. A61F 5/00

(52) U.S. Cl. 602/7

(57) ABSTRACT

The present disclosure relates to joint wraps that provide advantages over known wraps, particularly in terms of optimal efficacy, conformance of the wrap to the joint, and/or resilience of the wrap to deformation during use. The joint wraps are most preferably knee or elbow wraps. In particular, the present joint wraps include those comprising:

(a) a body portion, wherein the body portion creates a longitudinal axis; wherein the body portion comprises an aperture having a longest length and a widest width, wherein the longest length is substantially transverse to the longitudinal axis; and

(b) a body-facing surface and an outer surface, wherein each of the body-facing surface and outer surface extend along the length of the longitudinal axis.

The disclosure further relates to methods of treating pain selected from acute muscular, acute skeletal, acute referred, recurrent muscular, recurrent skeletal, recurrent referred, chronic muscular, chronic skeletal, chronic referred pain, and combinations thereof, comprising applying the foregoing joint wrap to the joint of a mammal in need of such treatment.

This application claims the benefit of U.S. Provisional Application Serial No. 60/424,200, filed on Nov. 6, 2002.

### FIELD OF THE INVENTION

The present invention relates to joint wraps suitable for mammalian use, having an aperture that is advantageously disposed to enhance efficacy, conformance of the wrap to the joint, and/or resilience of the wrap to deformation during use.

### BACKGROUND OF THE INVENTION

A common method of treating temporary or chronic pain is by application of heat to the afflicted area. Such heat treatments are used as a means of therapy for conditions which include aches, stiffness in muscles and joints, nerve pain, rheumatism, and the like. Typically, the method for relieving pain using heat treatments has been topical application of relatively high heat, e.g., greater than about 40° C. for a short period of time, e.g., from about twenty minutes to about one hour.

The mammalian knees and elbows, and particularly the human knee, are among the joints which are most vulnerable to overstress injury. While elastic compression bandages have been used to help stabilize knee movement during injury healing, heating pads, whirlpools, hot towels, hot water bottles, hot packs, and the like have been commonly used to apply heat to the joint to relieve the pain of joint injury. However, many of these devices are inconvenient for use on a regular and extended basis because the heat energy may not be immediately available when needed or released in a controllable manner. That is, many of these thermal units or devices do not provide long lasting heat and also do not maintain a consistent temperature over long periods of time. Proper positioning of the thermal energy also may not be maintainable during joint flexure. In general, the beneficial therapeutic effects from this administration of heat diminish after the heat source is removed.

Disposable heat packs based on iron oxidation, such as those described in U.S. Pat. Nos. 4,366,804; 4,649,895; 5,046,479 and Re. 32,026, are known. However, such devices have not been proven satisfactory because many of these devices are bulky, cannot maintain a consistent and controlled temperature, present difficulty staying in place during use, and/or have unsatisfactory physical dimensions that hinder their efficacy. Specifically, such devices cannot be easily incorporated into wraps that comfortably and reliably conform to various body contours, and hence, deliver inconsistent, inconvenient and/or uncomfortable heat application to the body.

With previous joint wraps such as knee wraps, it is conventional to construct a wide, oval aperture within the wrap, wherein such aperture is longitudinally aligned with the length of the wrap, for exposure of the patella (often referenced as a “knee cap”). It is believed that the aperture has conventionally been designed in this fashion would facilitate vertical movement of the knee, thus allowing for a comfortable fit. However, the present inventors have found that the longitudinal alignment of such an aperture, in any type of knee or elbow wrap, is not advantageous and may contribute to decreased efficacy, improper fit around the knee or elbow, and deformation of the wrap. For example, as described more particularly herein, the inventors have found that the function of the aperture is dependent upon the orientation of the longest length of such aperture.

Very recently, improved knee wraps have been described in such references as U.S. Pat. Nos. 5,728,057; 5,728,058; 5,860,945; and 6,048,326 and WO 98/29064. However, these references are not specific with regard to the precise disposal or orientation of the optional aperture. While the previously described knee wraps have provided significant and important advances in the art, the present inventors have discovered even further advances with respect to the aperture that are important for optimal efficacy, conformance to the joint (particularly the knee or elbow), and/or resilience to deformation.

The presently inventive knee wraps therefore provide further advantages relative to those provided in the art. These and other advantages of the knee wraps are described more particularly herein.

### SUMMARY OF THE INVENTION

The present invention is directed to joint wraps that provide advantages over known wraps, particularly in terms of optimal efficacy, conformance of the wrap to the joint, and/or resilience of the wrap to deformation during use. In particular, the present joint wraps include those comprising:

(a) a body portion, wherein the body portion creates a longitudinal axis; wherein the body portion comprises an aperture having a longest length and a widest width, wherein the longest length is substantially transverse to the longitudinal axis; and

(b) a body-facing surface and an outer surface, wherein each of the body-facing surface and outer surface extend along the length of the longitudinal axis.

The joint wraps are most preferably knee or elbow wraps.

The present invention is further directed to methods of treating pain selected from acute muscular, acute skeletal, acute referred, recurrent muscular, recurrent skeletal, recurrent referred, chronic muscular, chronic skeletal, chronic referred pain, and combinations thereof, comprising applying the foregoing joint wrap to the joint of a mammal in need of such treatment.

### BRIEF DESCRIPTION OF THE DRAWINGS

While the specification concludes with claims that particularly point out and distinctly claim the present invention, it is believed that the present invention is further understood from the following description of preferred embodiments, taken in conjunction with the accompanying drawings, wherein:

FIG. 1 is a top view of a preferred embodiment of the joint wraps of the present invention, wherein the joint wrap is a knee wrap, showing the aperture which has a longest length and a widest width, wherein the longest length is substantially transverse to the longitudinal axis of the wrap.

### DETAILED DESCRIPTION OF THE INVENTION

Various documents including, for example, publications and patents, are recited throughout this disclosure. All such documents are hereby incorporated by reference.

All percentages and ratios are calculated by weight unless otherwise indicated. All percentages and ratios are calculated based on the total composition unless otherwise indicated.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments and/or individual features are disclosed. As will be apparent to the ordinarily skilled practitioner, all combinations of such embodiments and features are possible and can result in preferred executions of the present invention.

The compositions herein may comprise, consist essentially of, or consist of any of the elements as described herein.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

As used herein, the term “elbow” includes the human elbow and the corresponding structures of other mammals.

As used herein, the term “knee” includes the human knee and the corresponding structures of other mammals.

As used herein, the term “mammal” means vertebrate mammals. Preferred mammals are humans and companion animals (e.g., domestic cats, dogs, horses, cows, or other similar animals). The most preferred mammals are humans.

As used herein, the term “plurality” with reference to a given noun means more than one, preferably more than two, more preferably more than three, and most preferably more than four units of the given noun.

### Joint Wraps and Methods of the Present Invention

The present joint wraps include those comprising:

(a) a body portion, wherein the body portion comprising a longitudinal axis; wherein the body portion comprises an aperture having a longest length and a widest width, wherein the longest length is substantially transverse to the longitudinal axis; and

(b) a body-facing surface and an outer surface, wherein each of the body-facing surface and outer surface extend along the length of the longitudinal axis.

The Aperture

The present joint wraps comprise a body portion that creates a longitudinal axis. The body portion may be in a variety of forms, for example, a looped wrap wherein the longitudinal axis runs through the length of the wrap and creates a continuous loop. In this type of embodiment, for example wherein the wrap is a knee wrap, the looped wrap is typically applied to the knee by positioning the wrap around the foot of the user and then moving the wrap to encircle the user's knee. In another embodiment, often preferred, the body portion is substantially planar, and may be looped around the joint to form a looped structure which is releasably fastened via means such as hook and loop fastening devices or other like devices (exemplary fastening means are described further herein below). Indeed, the form of the body portion, for example whether looped or substantially planar, is not critical to this invention. Rather it is the orientation, and optionally the dimensions, of the aperture described herein that is important to this invention.

The body portion comprises an aperture having a longest length and a widest width, wherein the longest length is substantially transverse to the previously described longitudinal axis. In the most preferred embodiment herein, the aperture is intended to be aligned with the user's patella (in accordance with the description herein below) and serves to help properly position the joint wrap during use.

By “substantially transverse to the longitudinal axis” it is meant that the axis running through the longest length of the aperture is at an angle of no more than about 20 degrees, preferably no more than about 10 degrees, and most preferably no more than about 5 degrees, relative to the lateral axis of the body portion (wherein the lateral axis is the axis which is perpendicular to the longitudinal axis). In the most preferred embodiment herein, the body portion comprises an aperture having a longest length and a widest width, wherein the longest length of the aperture is transverse to the longitudinal axis. By “transverse to the longitudinal axis” (without modification by the word “substantially”) it is meant that the axis running through the longest length of the aperture is at an angle of about 0 degrees, relative to the lateral axis of the body portion (again, wherein the lateral axis is the axis which is perpendicular to the longitudinal axis). Meaning, in this most preferred embodiment, the longest length of the aperture is perpendicular, or approximately perpendicular, to the longitudinal axis.

The present inventors have discovered that wherein the longest length of the aperture is substantially transverse to the longitudinal axis, or transverse to the longitudinal axis, the joint wraps exhibit optimized properties in terms of efficacy, conformance to the joint, and/or resilience to deformation. Indeed, without intending to be limited by theory, the present inventors have discovered that this aperture orientation allows accommodation of joint movement in the vertical direction. In particular, the inventors have discovered that the joint wrap exhibits greater flexibility and may more easily stretch and move with the joint upon movement. Moreover, and also surprisingly, the inventors discovered that this orientation of the aperture also allows accommodation of joint movement in the horizontal direction. Indeed, wherein such discovery is utilized, the wrap may not exhibit any appreciable deformation wherein the leg is extended, resulting in optimized conformance to the joint as well as enhanced overall efficacy of the wrap.

In a preferred embodiment of the present invention, wherein the joint wrap is in a relaxed state, the ratio of the longest length to the widest width is at least about 3:1, more preferably at least about 5:1, and most preferably at least about 6:1. In another preferred embodiment, wherein the joint wrap is in a relaxed state, the ratio of the longest length to the widest width is from about 3:1 to about 10:1, more preferably from about 5:1 to about 10:1, and most preferably from about 5:1 to about 8.5:1. As used herein, “relaxed state,” with reference to the joint wrap, means the condition of the joint wrap when the wrap is exposed only to gravitational forces. This embodiment is the result of the inventors' further discovery regarding the optimal overall shape, and/or dimensions, of the aperture. Again without intending to be limited by theory, the inventors have discovered that a relatively longer, but less wide, aperture (e.g., a “slit”) actually reduces the force necessary to impart flexibility of the aperture, and the overall joint wrap, upon body movement. Indeed, it has been discovered that the properly oriented, relatively narrow (as defined by the ratio of the longest length to the widest width of the aperture) aperture allows for a broader range of motion without significantly increasing the amount of binding force surrounding the joint area. As a result, the wrap is comfortable during wear since the binding force surrounding the joint does not significantly change over a broad range of motion.

Alternatively or additionally, in particularly preferred embodiments herein, the longest length of the aperture is typically from about 3 cm to about 15 cm, more preferably from about 5 cm to about 12 cm, and most preferably from about 6 cm to about 10 cm. Also alternatively or additionally, in particularly preferred embodiments herein, the widest width of the aperture is typically from about 0.1 cm to about 4 cm, more preferably from about 0.5 cm to about 3 cm, and most preferably from about 0.75 cm to about 2 cm.

Columnar Stays

Prior to describing a particularly preferred, but optional, embodiment of the present invention wherein the wraps comprise one or more columnar stays that are capable of delivering heat or cold to the user of the joint wrap, it is first helpful to describe other optional embodiments relating to the wraps. For example, in a preferred embodiment of the present invention, the joint wraps provide heat or cold to the user's joint. In a particularly preferred embodiment herein, the wraps comprise one or more thermal packs to provide such heating or cooling, preferably wherein the body portion comprises at least one of the thermal packs and most preferably wherein the body portion comprises all of the thermal packs (i.e., wherein the knee wrap comprises more than one thermal pack). The thermal pack may optionally comprise one or more thermic cells. Thermal packs and thermic cells which are heat cells are readily adaptable to the wraps of the present invention are described in, for example, U.S. Pat. Nos. 5,728,057; 5,728,058; 5,860,945; and 6,048,326 and WO 98/29064.

The thermic cells may comprise, as appropriate, exothermic or endothermic materials integrated into the wrap. While exemplary exothermic and endothermic materials are described herein below, these materials are commonly understood in the art and may be readily integrated into the present wraps. Again, heat cells comprising exothermic materials are described in, for example, U.S. Pat. Nos. 5,728,057; 5,728,058; 5,860,945; and 6,048,326 and WO 98/29064. Alternatively, the wraps may comprise one or more thermoresponsive materials, for example water. See e.g., U.S. Pat. No. 2,602,302. Wherein the wrap comprises a thermoresponsive material, the wrap may then be externally heated or cooled, as desired. As stated, different materials may be capable of satisfying the options set forth above. Such materials may include, but are not limited to, those materials described further herein.

With this as background, the optional but particularly preferable columnar stays may be described. In this preferred embodiment, the wraps comprise one or more of the foregoing thermic cells (each of which are described more particularly herein below), wherein at least a portion of the thermic cells are disposed to form a substantially columnar support which is substantially transverse, and most preferably transverse, to the longitudinal axis (referenced herein as columnar stays, wherein the term “substantially transverse” and “transverse” have already been defined herein with reference to the aperture).

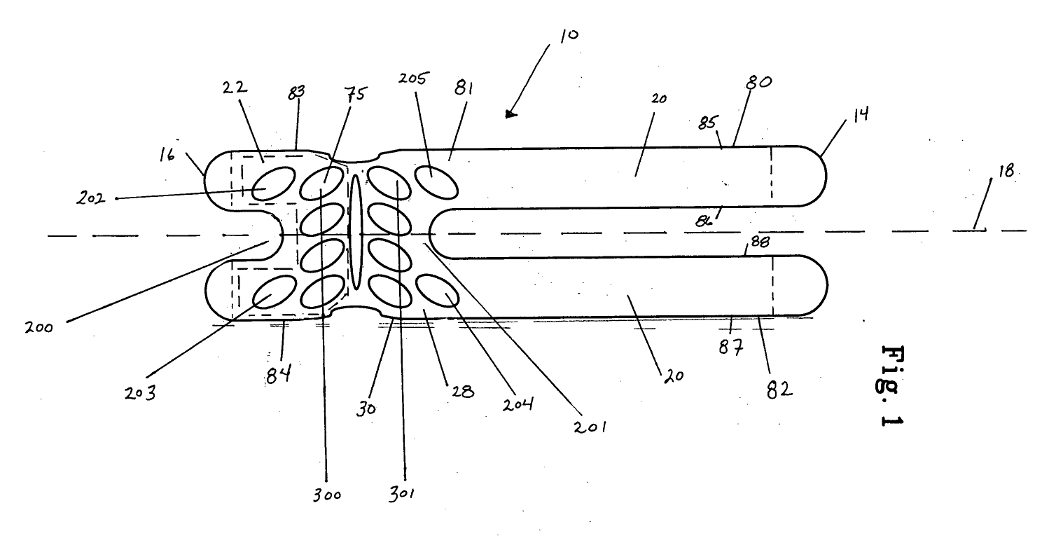

A joint wrap having a first columnar stay 300 and a second columnar stay 301 is illustrated in FIG. 1 herein, wherein each columnar stay is substantially parallel to the axis running through the longest length of the aperture. Using FIG. 1 as an example, the ordinarily skilled artisan will understand that the number of thermic cells making up each columnar stay may not be important. For example, wherein a given thermic cell is relatively large, the columnar design of such cell may be sufficient to create the stay. Alternatively, while FIG. 1 illustrates a columnar stay having 4 thermic cells, more or less cells may be utilized to create a given columnar stay. In addition, each cell making up a given columnar stay may be optionally offset from the other (e.g., in different proximities from the aperture relative to each other), provided the cells create a substantially columnar design. As such, the number and placement of thermic cells utilized for the columnar stay may be dependent upon a variety of factors, for example, the longest length of the aperture, the width of the body portion, the size or shape of each cell utilized, and the like.

With respect to this embodiment, the inventors have discovered that the integration of one or more of the foregoing columnar stays provides enhanced rigidity and structure to the joint wrap, without reliance on rigid or semi-rigid materials, or artificial stays (such as glue stays), as has been previously described in the art. See e.g., U.S. Pat. Nos. 5,728,057; 5,728,058; 5,860,945; and 6,048,326 and WO 98/29064 for discussion of rigid and semi-rigid materials, as well as artificial stays. The columnar stays described herein serve as resilient stiffeners to cause the wrap to maintain its flatness against the user's joint or surrounding body area. Indeed, the columnar stay described herein can serve two functions, i.e., delivery of heat or cold, as well as provision of enhanced rigidity and structure to the overall wrap. Of course, if so desired, the ordinarily skilled artisan may still utilize the foregoing rigid or semi-rigid materials, or glue or other artificial stays, without departing from the invention herein.

Compression Area

In another particularly preferred, but optional, embodiment of the present invention, the present joint wraps are free of thermic cells which are fully disposed in the compression area. As used herein the term “compression area,” means that area of the body of the mammalian user that is subject to relatively high motion during joint flexure. For example, with reference to the human knee, the compression area is that area that is offset approximately 45 degrees from the center of the human patella, or the “sides of the knee.” Therefore, joint wraps which are free of thermic cells which are fully disposed in the compression area are free of cells which would otherwise fully contact the compression area during ordinary use of the wrap. In this embodiment, the inventors have discovered that placement of cells in this area is often undesirable due to the relatively high motion upon flexure, which can trigger undesirable exothermic or endothermic reactions that will result in substantial temperature differences between these cells and the cells proximal to relatively lower motion areas.

To illustrate, FIG. 1 indicates the joint wrap having an aperture that is transverse to the longitudinal axis and columnar stays 300 and 301 disposed on either side of the aperture, wherein the joint wrap is free of thermic cells which are fully disposed in the compression area. In this FIG. 1, the compression area is collectively indicated as first compression area 200 and second compression area 201 (in this illustration, the joint wrap has actually been designed to diminish any presence of the wrap itself in the compression area, thereby creating a void space). Because thermic cells 202, 203, 204, and 205 are positioned in a manner such that they are not fully disposed in a compression area, but rather only are only minimally capable of contact with a compression area during ordinary use of the wrap, the wrap of this example is free of thermic cells which are fully disposed in the compression area.

Other Optional Embodiments of the Present Invention

Other optional embodiments of the present invention will be readily apparent to the ordinarily skilled artisan. As has been stated, the joint wraps described herein may be optionally but readily adapted to any of the descriptions set forth in U.S. Pat. Nos. 5,728,057; 5,728,058; 5,860,945; and 6,048,326 and WO 98/29064.

In a particularly preferred embodiment of the present invention, the present joint wraps are a unified structure. However, one of ordinary skill will readily understand that certain components of the wrap may be releasably attached to other components of the wrap. For example, fastening systems may be releasably attached to the remainder of the wrap.

In a further particularly preferred embodiment of the present invention, the present joint wraps are disposable. As used herein, the term “disposable” means that, while the joint wraps of the present invention may be stored in a resealable, substantially air-impermeable container and reapplied to the user's body as often as required for the relief of pain, they are intended to be disposed of after the wrap is functional, for example, after the optional heat or cold source is fully expended.

In an optional, but particularly preferred embodiment herein, the joint wrap comprises a first end and a second end, wherein the body portion is disposed between the first end and the second end. In this embodiment, the joint wrap is a unified structure comprising a first end, a second end, and the body portion, wherein the body portion is disposed between the first end and second end; wherein the disposition of the first end, the second end, and the body portion create the longitudinal axis; and wherein the body portion comprises an aperture having a longest length and a widest width, wherein the longest length is substantially transverse to the longitudinal axis.

To illustrate, and referring now to the drawings, and more particularly to FIG. 1, there is shown a preferred embodiment of the present invention, which provides a joint wrap, generally indicated as 10, wherein the joint wrap is a knee wrap. The knee wrap comprises a longitudinal axis 18. The wrap has a first end 14 and a second end 16 and elastic portions 20 capable of being stretched along longitudinal axis 18. The wrap has a length, as measured in a direction parallel to longitudinal axis 18 from first end 14 to second end 16, when in a relaxed state or a stretched state, which is great enough to encircle a user's knee, such that first end 14 overlaps second end 16 when the wrap is positioned around user's knee.

Again to illustrate, and again referring to the drawings, the wrap has a body-facing surface 28 and an outer surface 30, wherein each of the body-facing surface and outer surface extend along the length of the longitudinal axis (i.e., each surface extends from first end 14 to second end 16).

The wrap further comprises body portion 81 which is disposed between the first end and the second end. Again by way of illustration only, body portion 81 has a first edge 83 and a second edge 84. The distance between first edge 83 and second edge 84 measured in a direction transverse to the longitudinal axis 18 is the width of body portion 81. When utilized, upper strap portion 80 has a first edge 85 and a second edge 86. The distance between first edge 85 and second edge 86 measured in a direction transverse to longitudinal axis 18 is the width of upper strap portion 80. Also when utilized, lower strap portion 82 has a first edge 87 and a second edge 88. The distance between first edge 87 and second edge 88 measured in a direction transverse to longitudinal axis 18 is the width of lower strap portion 82.

Preferably, the longest width of body portion 81 is from about 12 cm to about 25 cm, more preferably from about 13 cm to about 23 cm and most preferably from about 13 cm to about 18 cm. The widths of upper strap portion 80 and lower strap portion 82 are each, independently, typically less than the width of body portion 81, and preferably from about 2.5 cm to about 13 cm, more preferably from about 3 cm to about 8 cm, and most preferably form about 4 cm to about 7 cm.

In another preferred embodiment herein, the joint wrap comprises one or more elastic portions stretchable along the longitudinal axis. Use of such elastic portions is particularly beneficial for ensuring proper fit among a variety of users having differing leg or arm and/or knee or elbow measurements (as applicable) or other characteristics. As used herein, the word “elastic” with reference to the elastic portion refers to that property of a material whereby the material, when subjected to a tensile force, will stretch or expand in the direction of the force and will substantially return to its original untensioned dimension upon removal of the force. More specifically, the term “elastic” is intended to mean a directional property wherein an element or structure has a recovery to within about 10% of its original length Lₒ after being subjected to a percent strain ε_{%} of greater than 50%. As used herein, percent strain ε_{%} is defined as:

ε_{%}=[(L_{f}−Lₒ)/Lₒ]*100

Where

L_{f}=Elongated Length

Lₒ=Original Length

For consistency and comparison, the recovery of an element or structure is preferably measured 30 seconds after release from its elongated length L_{f}. All other elements or structures will be considered inelastic if the element or structure does not recover to within about 10% of its original length Lₒ within 30 seconds after being released from a percent strain ε_{%} of 50%. Inelastic elements or structures would also include elements or structures which fracture and/or permanently/plastically deform when subjected to a percent strain ε_{%} of 50%.

Elastic portions may be independently selected from natural or synthetic rubber, or any number of polymeric materials which are capable of elongation and recovery. Suitable materials include, but are not limited to, styrene block copolymers, rubber, LYCRA™, KRAYTON™, polyethylene including metallocene catalyst PE, foams including polyurethane and polyesters, and the like. The elastic portions may be independently in the form of films, strands, scrims, ribbons, tapes, structural elastic-like film, and the like. A particularly suitable material for one or more of the elastic portions is an elastic scrim available as X50020 from Conwed Plastics, Minneapolis, Minn.

To improve the elastic performance of the wrap, the elastic portions may be subjected to any activation process after assembly and prior to use. This activation process stretches and permanently deforms on a very small scale the nonelastic layers of the wrap within the elastic portion. This activation process allows the elastic to stretch or expand in the direction of an applied force and essentially return to original dimensions upon removal of the force, unencumbered by the nonelastic layers of the elastic.

Alternatively, the elastic portions may be assembled while the elastic is held in an extended state. After assembly, the elastic is allowed to return to a relaxed state causing the nonelastic layers to fold and buckle creating rugosities. Subsequent stretching of the elastic will result in the unfolding of these rugosities.

The outer surface of the joint wrap may be made from any number of different materials. For example, such materials may include, but are not limited to, woven and knit fabrics, carded nonwovens, spunbond nonwovens, and the like may be utilized. A material that has been found to be particularly suitable for the body-facing surface and outer surface is a carded thermally bonded nonwoven of polypropylene with a basis weight of 32 g/m² (27 grams per square yard (gsy)). This material is available as grade #6520, commercially available from Polymer Group Incorporated of Landisville, N.J. In particularly preferred embodiments, the outer surface comprises any warp or weft knit fabric having loops knitted into the fabric, or nonwovens or nonwoven laminates capable of supporting hook attachment. In a preferred embodiment herein, at least a portion of the outer surface is permeable to air, such that the exothermic or endothermic composition, when used, may be appropriately activated.

In another, preferred, embodiment herein, a stiffening material is adhered to the outer surface of the joint wrap. Stiffening materials may be chosen from any number of suitable materials which provide rigidity in a direction transverse to the longitudinal axis. Suitable materials include, but are not limited to, wovens, knits, carded nonwovens, spunbound nonwovens, meltblown nonwovens, combinations thereof, and the like. These fabrics may be made of either natural or synthetic fibers including, but not limited to, polypropylene, polyester, nylon, rayon, cotton, cellulose, combinations thereof, and the like. These materials may be post processed to increase their stiffness. This post processing may include calandering, embossing, bonding, and the like. Particular materials for such stiffening layer includes spunbound/meltblown/spunbound (SMS) laminates, for example available as Grade #W502FWA, commercially available from Polymer Group Incorporated of Landisville, N.J.

One or more thermal packs may also be utilized herein, and may be integrated between the outer surface and the body-facing surface. In a particularly preferred embodiment herein, the stiffening layer is adhered to both the outer surface and the thermal pack, such that the stiffening layer is integrated between the outer surface and the thermal pack. As has been stated, the thermal packs may contain an exothermic or endothermic composition, as desired, or any of a variety of thermoresponsive materials such as water.

In one particular embodiment herein, the thermal packs previously described herein may have at least one continuous layer of a material which preferably exhibits specific thermophysical properties, and optionally a plurality of individual thermic cells which preferably comprise an exothermic or endothermic composition, as applicable, spaced apart and fixed within or to the structure of the disposable thermal pack. The cells are a unified structure, comprising the exothermic or endothermic composition, enclosed within two layers, wherein at least one layer may be oxygen permeable, capable of providing long lasting heating or cooling with improved temperature control, and having specific physical dimensions and fill characteristics. These cells can be used as individual temperature control units, or in a thermal pack comprising a plurality of individual cells which can also be easily incorporated into disposable body wraps, pads, and the like. Thermal packs and body wraps incorporating thermal packs adapt to a wide variety of body contours, thus providing consistent, convenient, and comfortable heating or cooling application.

For example, again referring to FIG. 1, the joint wraps may comprise one or more thermal packs 22 arranged in a pattern. Thermal pack 22 is typically constructed by forming a pocket in a base material. The pocket is filled with an exothermic composition. After filling the pocket, a cover material is placed over the pocket and heat sealed to the base material around the periphery of the pocket, encapsulating the exothermic composition in heat cell 75.

Preferred spacing between heat cells, thereby creating certain drape characteristics, is described in considerable detail in U.S. Pat. Nos. 5,728,057; 5,728,058; 5,860,945; and 6,048,326 and WO 98/29064.

Endothermic and exothermic compositions are readily known in the art. The use of exothermic compositions is particularly preferred by the inventors. Exothermic compositions may comprise any composition capable of providing heat. Preferably, the exothermic composition comprises a particulate mix of chemical compounds that undergo an oxidation reaction during use. Alternatively, the exothermic composition may also be formed into agglomerated granules, direct compacted into compaction articles such as granules, pellets, tablets, and/or slugs, and mixtures thereof. The mix of compounds typically comprises iron powder, carbon, a metal salt(s), and water. Mixtures of this type, which react when exposed to oxygen, provide heat for several hours.

Suitable sources for iron powder include cast iron powder, reduced iron powder, electrolytic iron powder, scrap iron powder, pig iron, wrought iron, various steels, iron alloys, and the like and treated varieties of these iron powders. There is no particular limitation to their purity, kind, and other properties, so long as it can be used to produce heat-generation with electrically conducting water and air. Typically, the exothermic composition comprises from about 30% to about 80% iron powder, more preferably from about 50% to about 70% iron powder, all by weight of the exothermic composition.

Carbonaceous material selected from the group consisting of activated carbon, non-activated carbon, and mixtures thereof may be used in the exothermic compositions. Active carbon prepared from coconut shell, wood, charcoal, coal, bone coal, and the like are useful, but those prepared from other raw materials such as animal products, natural gas, fats, oils and resins are also useful in the particulate exothermic composition optionally used herein. There is no limitation to the kinds of active carbon used, however, the preferred active carbon has superior water holding capabilities and the different carbons may be blended to reduce cost. Therefore, mixtures of the above carbons are useful in the present invention as well. Typically, the composition comprises from about 3% to about 25% carbonaceous material, more preferably from about 8% to about 20% carbonaceous material, and most preferably from about 9% to about 15% carbonaceous material, all by weight of the composition.

The metal salts useful in the particulate exothermic composition include sulfates such as ferric sulfate, potassium sulfate, sodium sulfate, manganese sulfate, magnesium sulfate; and chlorides such as cupric chloride, potassium chloride, sodium chloride, calcium chloride, manganese chloride, magnesium chloride and cuprous chloride. Also, carbonate salts, acetate salts, nitrates, nitrites and other salts can be used. In general, several suitable alkali, alkaline earth, and transition metal salts exist which can also be used, alone or in combination, to sustain the corrosive reaction of iron. The preferred metal salts are sodium chloride, cupric chloride, and mixtures thereof. Typically, the exothermic composition comprises from about 0.5% to about 10%, more preferably from about 1.0% to about 5% by weight, metal salts, all by weight of the exothermic composition.

The water used in the particulate exothermic composition may be from any appropriate source. There is no particular limitation to its purity, kind, and the like. Typically, the exothermic composition comprises from about 1% to about 40%, more preferably from about 10% to about 30%, water, all by weight of the exothermic composition.

Additional water-holding materials may also be optionally added as appropriate. Useful additional water-holding materials include vermiculite, porous silicates, wood powder, wood flour, cotton cloth having a large amount of fluffs, short fibers of cotton, paper scrap, vegetable matter, super absorbent water-swellable or water-soluble polymers and resins, carboxymethylcellulose salts, and other porous materials having a large capillary function and hydrophilic property can be used. Typically, wherein the exothermic composition comprises the water-holding materials, the exothermic composition comprises from about 0.1% to about 30%, more preferably from about 0.5% to about 20% by weight, and most preferably from about 1% to about 10%, water-holding materials, all by weight of the exothermic composition.

Other additional components include agglomeration aids such as gelatin, natural gums, cellulose derivatives, cellulose ethers and their derivatives, starch, modified starches, polyvinyl alcohols, polyvinylpyrrolidone, sodium alginates, polyols, glycols, corn syrup, sucrose syrup, sorbitol syrup and other polysaccharides and their derivatives, polyacrylamides, polyvinyloxoazolidone, and maltitol syrup; dry binders such as maltodextrin, sprayed lactose, co-crystallized sucrose and dextrin, modified dextrose, sorbitol, mannitol, microcrystalline cellulose, microfine cellulose, pre-gelatinized starch, dicalcium phosphate, and calcium carbonate; oxidation reaction enhancers such as elemental chromium, manganese, or copper, compounds comprising said elements, or mixtures thereof; hydrogen gas inhibitors such as inorganic or organic alkali compounds or alkali weak acid salts including sodium hydroxide, potassium hydroxide, sodium hydrogen carbonate, sodium carbonate, calcium hydroxide, calcium carbonate, and sodium propionate; fillers such as natural cellulosic fragments including wood dust, cotton linter, and cellulose, synthetic fibers in fragmentary form including polyester fibers, foamed synthetic resins such as foamed polystyrene and polyurethane, and inorganic compounds including silica powder, porous silica gel, sodium sulfate, barium sulfate, iron oxides, and alumina; and anti-caking agents such as tricalcium phosphate and sodium silicoaluminate. Such components also include thickeners such as cornstarch, potato starch, carboxymethylcellulose, and alpha-starch, and surfactants such as those included within the anionic, cationic, nonionic, zwitterionic, and amphoteric types. The preferred surfactant, if used however, is nonionic. Still other additional components which may be added to the particulate exothermic compositions of the present invention, as appropriate, include extending agents such as metasilicates, zirconium, and ceramics.

Preferably at least 50%, more preferably 70%, even more preferably 80% and most preferably 90% of all of the particles, by weight of the exothermic composition, have a mean particle size of less than 200 microns, preferably less than 150 microns.

The above-mentioned components of the composition are blended using conventional blending techniques. Suitable methods of blending these components are described in detail in U.S. Pat. No. 4,649,895.

Alternatively to the above described particulate exothermic composition, the exothermic composition may be formed into agglomerated granules, direct compacted into compaction articles such as granules, pellets, tablets, and/or slugs, and mixtures thereof, which may be referenced as agglomerated pre-compaction compositions. As used herein, the term “agglomerated pre-compaction composition” means the mixture of dry powdered ingredients, comprising iron powder, carbonaceous powder, metal salt(s), water-holding agent(s), agglomeration aid(s), and dry binder(s) prior to direct compaction. As used herein, the term “direct compacted” or “direct compaction” means a dry powder mixture is or has been blended, compressed, and formed into pellets, tablets, or slugs without the use of typical wet binders/solutions to adhere the particulate(s) together. Alternatively, the dry powder mixture is, or has been, blended and roll compacted or slugged, followed by milling and screening, creating directly compacted granules. Direct compaction may also be known in the art as dry compaction. Other suitable methods of making tablets and/or slugs are described in detail in Chapter 89, “Oral Solid Dosage Forms,” Remington's Pharmaceutical Sciences, 18^{th} Edition, (1990), pp. 1634-1656.

The thermic cells can have any geometric shape, e.g., disk, triangle, square, cube, rectangle, cylinder, ellipsoid and the like, all or none of which may contain a hole through the middle or other reservoir.

The preferred shape of the cell comprises an ellipsoid geometry. In a preferred embodiment, the ellipsoid shapes may have a width at its widest point of from about 0.15 cm to about 20 cm, preferably from about 0.3 cm to about 10 cm, more preferably from about 0.5 cm to about 5 cm, most preferably from about 1 cm to about 3 cm, a height at its highest point of from about 0.1 cm to about 5 cm, preferably from about 0.2 cm to about 1 cm, more preferably from about 0.2 cm to about 0.8 cm, and most preferably from about 0.2 cm to about 0.7 and a length at its longest point of from about 0.5 cm to about 20 cm, preferably from about 1 cm to about 15 cm, more preferably from about 1 cm to about 10 cm, most preferably from about 3 cm to about 5 cm.

Alternatively, cells having geometric shapes other than an ellipsoid shape, preferably a disk shape may be used. The preferred disk shapes preferably have a cell diameter of from about 0.2 cm to about 10 cm, preferably from about 0.5 cm to about 8 cm, more preferably from about 1 cm to about 5 cm, and most preferably from about 1.5 cm to about 3 cm. Cells preferably have a height of from about 0.1 cm to about 1 cm, preferably from greater than about 0.1 cm to about 0.9 cm, more preferably from greater than about 0.2 cm to about 0.8 cm, and most preferably from greater than about 0.2 cm to about 0.7 cm.

The compaction articles are preferably compressed to a mechanical strength which is capable of withstanding the shocks of handling in their manufacture, packing, shipping, and dispensing. The compaction articles are typically compressed to a density of greater than about 1 g/cm³, preferably from about 1 g/cm³ to about 3 g/cm³, more preferably from about 1.5 g/cm³ to about 3 g/cm³, and most preferably from about 2 g/cm³ to about 3 g/cm³.

In a preferred embodiment, the ratio of fill volume to cell volume of a given cell is from about 0.7 to about 1.0, preferably from about 0.75 to about 1.0, more preferably from about 0.8 to about 1.0, even more preferably from about 0.85 to about 1.0, and most preferably from about 0.9 to about 1.0. As used herein, the term “fill volume” means the volume of the particulate composition or the compacted, water-swelled, heating element in the filled cell. As also used herein, the term “cell volume” means the fill volume plus the void volume of the cell. As also used herein, the term “void volume” means the volume of the cell left unfilled by the particulate composition or the compacted, water-swelled, heating element in a finished heat cell, not including the unfilled space within a tablet comprising a hole or reservoir, in a finished heat cell, measured without differential pressure in the cell and without additional stretching or deformation of the substrate material.

Oxygen permeability, allowing enhancement of the exothermic or endothermic reaction, may optionally be provided by selecting materials for the previously described base material and/or cover material that have the specifically desired permeability properties. The desired permeability properties may be provided by microporous films or by films which have pores or holes formed therein. The formation of these holes/pores may be via extrusion cast/vacuum formation or by hot needle aperturing. Oxygen permeability can also be provided in the present invention by perforating at least one of the base material and cover material with aeration holes using, for example, an array of pins having tapered points and diameters of from about 0.2 mm to about 2 mm, preferably from about 0.4 mm to about 0.9 mm. The array of pins is patterned such that the base material and/or cover material are perforated by from about 10 to about 30 pins per square centimeter. Alternatively, after the base material and cover material have been bonded together, enclosing the exothermic composition in the pocket between them, at least one side of the cell may be perforated with aeration holes using, for example, at least one pin, preferably an array of from about 20 to about 60 pins having tapered points and diameters of from about 0.2 mm to about 2 mm, preferably from about 0.4 mm to about 0.9 mm. The pins are pressed through one side of the base material and/or cover material to a depth of from about 2% to about 100%, preferably from about 20% to about 100%, and more preferably from about 50% to about 100% into the exothermic composition. This hole configuration provides an oxygen diffusion into the cell during oxidation of the endothermic or exothermic composition of from about 0.01 cc O₂/min./5 cm² to about 15.0 cc O₂/min./5 cm² (at 21° C., 1 ATM), preferably from about 0.9 cc O₂/min./5 cm² to about 3 cc O₂/min./5 cm² (at 21° C., 1 ATM).

The velocity, duration, and temperature of the thermogenic oxidation reaction of the endothermic or exothermic composition can be controlled as desired by changing the area of contact with air, more specifically, by changing the oxygen diffusion/permeability.

The body-facing surface (as exemplified by body-facing surface 28) of the joint wrap may be made from any number of different materials. For example, such materials may include, but are not limited to, woven and knit fabrics, carded nonwovens, spunbond nonwovens, and the like may be utilized. A material that has been found to be particularly suitable for the body-facing surface and outer surface is a carded thermally bonded nonwoven of polypropylene with a basis weight of 32 g/m (27 grams per square yard (gsy)). This material is available as grade #6520, commercially available from Polymer Group, Incorporated of Landisville, N.J.

In a particularly preferred embodiment, the wrap comprises the outer surface, the stiffening layer, the thermal packs comprising one or more cells, and the body-facing surface. In addition to these layers, further optional bulking layers may be utilized for the knee wrap. Bulking layers may comprise any number of different materials which include, but are not limited to, woven and knit fabrics, formed films, carded nonwovens, spunbond nonwovens, and the like.

Attachment of the various layers of the joint wrap described herein may be achieved by any number of attachment means known in the art. These include, but are not limited to, hot melt adhesive including spiral sprays, meltblown, control coat, and the like, latex adhesives applied via spray, printing, gravure, and the like, thermal bonding, ultrasonic, pressure bonding, and the like. Preferably, an adhesive layer is used. One particular method includes a hot melt adhesive available as 70-4589 from National Starch and Chemical Co., Bridgewater, N.J., applied via a hot melt system.

The joint wraps herein may also optionally comprise any of a variety of fastening means, utilized to secure the wrap around the user's joint. Fastening means have been previously described, for example, in U.S. Pat. Nos. 5,728,057; 5,728,058; 5,860,945; and 6,048,326 and WO 98/29064. Additionally, while the use of columnar stays as described herein has been found to be beneficial to the present invention, other optional stays may be embedded, typically transverse to the longitudinal axis 18. Various of these stays are also described in U.S. Pat. Nos. 5,728,057; 5,728,058; 5,860,945; and 6,048,326 and WO 98/29064. In addition, all other components not explicitly described herein but described in U.S. Pat. Nos. 5,728,057; 5,728,058; 5,860,945; and 6,048,326 and WO 98/29064 may also be readily incorporated in the joint wraps of the present invention.

The joint wraps of the present invention may also optionally incorporate a component, such as a separate substrate layer or incorporated into at least one of the continuous layers, comprising active aromatic compounds, non-active aromatic compounds, pharmaceutical actives or other therapeutic agents, and mixtures thereof, to be delivered through the skin. Such active aromatic compounds include, but are not limited to, menthol, camphor, and eucalyptus. Such non-active aromatic compounds include, but are not limited to, benzaldehyde, citral, decanal, and aldehyde. Such pharmaceutical actives/therapeutic agents include, but are not limited to antibiotics, vitamins, antiviral agents, analgesics, anti-inflammatory agents, antipruritics, antipyretics, anesthetic agents, antifungals, antimicrobials, and mixtures thereof. The disposable thermal knee wraps may also comprise a separate substrate layer, or incorporated into at least one of the continuous layers, a self-adhesive component and/or a sweat-absorbing component.

The finished joint wraps are typically packaged in a secondary package. An air-impermeable package may be used to prevent an oxidation reaction from occurring until desired as described in U.S. Pat. No. 4,649,895. Alternatively, wherein exothermic or endothermic compositions are used, other means may also be used to prevent an oxidation reaction from occurring before desired, such as air impermeable removable adhesive strips placed over the aeration holes in the cells such that, when the strips are removed, air is allowed to enter the cells, thus activating the oxidation reaction of the iron powder.

The present invention further comprises a method for treating acute, recurrent, and/or chronic joint pain, including muscular, skeletal, and/or referred joint pain, of a person suffering such pain by applying the present joint wrap to the joint of a person suffering such pain. The joint is preferably the elbow or knee, most preferably knee.

The method comprises maintaining a skin temperature to the joint of a person suffering such pain of from about 32° C. to about 50° C., preferably from about 32° C. to about 45° C., more preferably from about 32° C. to about 42° C., most preferably from about 32° C. to about 39° C., still most preferably from about 32° C. to about 37° C., preferably by applying the above described joint wraps to the joint of a person suffering such pain, for from about twenty seconds to about twenty-four hours, preferably from about twenty minutes to about twenty hours, more preferably from about four hours to about sixteen hours, most preferably from about eight hours to about twelve hours, wherein the maximum skin temperature and the length of time of maintaining the skin temperature at the maximum skin temperature may be appropriately selected by a person needing such treatment, such that the desired therapeutic benefits are achieved, without any adverse events, such as skin burns which may be incurred by using a high temperature for a long period of time. It is preferred that such treatment substantially relieves acute, recurrent, and/or chronic joint pain, including skeletal, muscular, and/or referred joint pain, of a person having such pain and to substantially prolong relief, for at least about two hours, preferably for at least about 8 hours, more preferably for at least about sixteen hours, most preferably for at least about one day, still most preferably for at least about three days, from such pain, even after the heat source is removed from the joint of the user.

While particular embodiments of the present invention have been illustrated and described, it will be obvious to those skilled in the art that various changes and modifications may be made without departing from the spirit and scope of the invention, and it is intended to cover in the appended claims all such modifications that are within the scope of the invention.

What is claimed is:

1. A joint wrap comprising:
(a) a body portion, wherein the body portion creates a longitudinal axis; wherein the body portion comprises an aperture having a longest length and a widest width, wherein the longest length is substantially transverse to the longitudinal axis; and
(b) a body-facing surface and an outer surface, wherein each of the body-facing surface and outer surface extend along the length of the longitudinal axis.

2. The joint wrap according to claim 1 wherein when the joint wrap is in a relaxed state, the ratio of the longest length to the widest width is at least about 3:1.

3. The joint wrap according to claim 2 comprising one or more elastic portions stretchable along the longitudinal axis.

4. The joint wrap according to claim 3 further comprising a first end and a second end, wherein the body portion is disposed between the first end and second end; wherein the disposition of the first end, the second end, and the body portion create the longitudinal axis.

5. The joint wrap according to claim 4 further comprising one or more thermal packs, wherein the body portion comprises at least one of the thermal packs.

6. The joint wrap according to claim 5 wherein the body portion comprises one or more strap portions, wherein each strap portion comprises one of the elastic portions.

7. The joint wrap according to claim 6 wherein each thermal pack comprises one or more thermic cells, wherein the thermic cells comprise a composition selected from the group consisting of endothermic compositions and exothermic compositions.

8. The joint wrap according to claim 7 wherein when the joint wrap is in a relaxed state, the ratio of the longest length to the widest width is at least about 5:1.

9. The joint wrap according to claim 8 wherein the thermic cells are heat cells comprising an exothermic composition.

10. The joint wrap according to claim 9 further comprising a fastening system.

11. The joint wrap according to claim 10 wherein when the joint wrap is in a relaxed state, the ratio of the longest length to the widest width is from about 5:1 to about 8.5:1.

12. The joint wrap according to claim 11 wherein the exothermic composition comprises:
(a) from about 30% to about 80% of iron powder, by weight of the exothermic composition; and
(b) from about 3% to about 25% of carbonaceous material selected from the group consisting of activated carbon, non-activated carbon, and mixtures thereof, by weight of the exothermic composition.

13. The joint wrap according to claim 12 wherein at least a portion of the outer surface is permeable to air.

14. The joint wrap according to claim 4 wherein the body portion comprises one or more thermic cells, wherein at least a portion of the cells are disposed to form a substantially columnar support which is substantially transverse to the longitudinal axis.

15. The joint wrap according to claim 7 wherein at least a portion of the thermic cells are disposed to form a substantially columnar support which is substantially transverse to the longitudinal axis.

16. The joint wrap according to claim 10 wherein at least a portion of the heat cells are disposed to form a substantially columnar support which is substantially transverse to the longitudinal axis.

17. The joint wrap according to claim 11 further comprising a stiffening layer.

18. The joint wrap according to claim 7 which is free of thermic cells which are fully disposed in the compression area.

19. The joint wrap according to claim 10 which is free of thermic cells which are fully disposed in the compression area.

20. A method of treating pain selected from the group consisting of acute muscular, acute skeletal, acute referred, recurrent muscular, recurrent skeletal, recurrent referred, chronic muscular, chronic skeletal, chronic referred pain, and combinations thereof, comprising applying the joint wrap according to claim 1 to the joint of a mammal in need of such treatment.

21. The method according to claim 20 wherein such treatment provides to the mammal a skin temperature of from about 32° C. to about 50° C. for a time period of from about 20 seconds to about twenty-four hours.

22. The method according to claim 21 wherein the skin temperature is from about 32° C. to about 43° C.

23. The method according to claim 22 wherein the time period is from about 1 hour to about twenty-four hours.
